(19) United States
(12) Patent Application Publication
(10) Pub. No.: US 2026/0076699 A1
(43) Pub. Date: Mar. 19, 2026

(54) MEDICAL DEVICE AND METHOD FOR MANUFACTURING MEDICAL DEVICE

(71) Applicant: ASAHI INTECC CO., LTD., Seto-shi (JP)

(72) Inventors: Issei ITO, Seto-shi (JP); Kanako NISHIO, Seto-shi (JP)

(73) Assignee: ASAHI INTECC CO., LTD., Seto-shi (JP)

(21) Appl. No.: 19/330,258

(22) Filed: Sep. 16, 2025

(30) Foreign Application Priority Data
Sep. 18, 2024 (JP) 2024-160685

(51) Int. Cl. A61B 17/221; A61B 17/00; A61B 90/00; A61M 25/09

(52) U.S. Cl. CPC A61B 17/221; A61B 2017/00526; A61B 2017/2212; A61B 2090/3966; A61M 25/09

(57) ABSTRACT

A medical device includes a main body part and a leading portion including loop-shaped loop portion that is formed by a coil. The coil is formed by a spirally wound wire rod. The leading portion is connected to a distal end of the main body part and can enter a lesion.

### CROSS REFERENCE TO RELATED APPLICATIONS

This is a Paris Convention application based on Japanese Patent Application JP 2024-160685. The disclosure of the prior application is hereby incorporated by reference herein in its entirety.

### TECHNICAL FIELD

The technology disclosed herein relates to a medical device and a method for manufacturing the medical device.

### BACKGROUND ART

Guide wires are used to treat stenosis sites or occlusion sites (hereinafter referred to as lesion) in blood vessels. Known guide wires include an elongate member. The elongate member includes a first portion having a first diameter and a second portion having a second diameter, and the second portion forms a loop (see, for example, Patent Document 1).

### CITATION LIST

Patent Document 1: Japanese Translation of PCT International Publication 2006-507899

### SUMMARY

### Technical Problem

Known guide wires leave room for improvement in safety.

This specification discloses a technology capable of solving the above-described issue.

### Solution to Problem

A medical device is disclosed herein comprising a main body part and a leading portion including a loop-shaped loop portion that is formed by a coil. The coil is formed by a spirally wound wire rod. The leading portion is connected to a distal end of the main body part and is configured to enter a lesion.

### BRIEF DESCRIPTION OF DRAWINGS

FIG. 1 is a plan view of a guide wire according to a first embodiment.

FIG. 2 is a side view of the guide wire according to the first embodiment.

FIG. 3 is a sectional view of the guide wire according to the first embodiment taken along the line III-III of FIG. 2.

FIG. 4 is a partially enlarged sectional view illustrating the inside of a frame F of FIG. 3 in an enlarged manner.

FIG. 5 is a partially enlarged sectional view illustrating the inside of a circle R of FIG. 4 in an enlarged manner.

FIG. 6 is a partially enlarged perspective view of a wire rod used for a coil according to the first embodiment.

FIG. 7 is a flowchart illustrating an example of a method for manufacturing the guide wire according to the first embodiment.

FIG. 8 is an explanatory view illustrating an example of the method for manufacturing the guide wire according to the first embodiment.

FIG. 9 is an explanatory view illustrating an example of the method for manufacturing the guide wire according to the first embodiment.

FIG. 10 is an explanatory view illustrating an example of the method for manufacturing the guide wire according to the first embodiment.

FIG. 11 is an explanatory view illustrating an example of a treatment method using the guide wire according to the first embodiment.

FIG. 12 is an explanatory view illustrating an example of the treatment method using the guide wire according to the first embodiment.

FIG. 13 is a partially enlarged sectional view illustrating a leading portion and a portion in the vicinity thereof in a guide wire according to a second embodiment, which is cut at the same position as the line III-III of FIG. 2.

FIG. 14 is a flowchart illustrating an example of a method for manufacturing the guide wire according to the second embodiment.

FIG. 15 is an explanatory view illustrating an example of the method for manufacturing the guide wire according to the second embodiment.

FIG. 16 is an explanatory view illustrating an example of the method for manufacturing the guide wire according to the second embodiment.

FIG. 17 is a sectional view illustrating a guide wire according to a third embodiment, which is cut at the same position as the line III-III of FIG. 2.

FIG. 18 is a flowchart illustrating an example of a method for manufacturing the guide wire according to the third embodiment.

FIG. 19 is an explanatory view illustrating an example of the method for manufacturing the guide wire according to the third embodiment.

FIG. 20 is an explanatory view illustrating an example of the method for manufacturing the guide wire according to the third embodiment.

FIG. 21 is an explanatory view illustrating an example of the method for manufacturing the guide wire according to the third embodiment.

FIG. 22 is an explanatory view illustrating an example of the method for manufacturing the guide wire according to the third embodiment.

FIG. 23 is a partially enlarged sectional view illustrating a leading portion and a portion in the vicinity thereof in a guide wire according to a fourth embodiment, which is cut at the same position as the line III-III of FIG. 2.

FIG. 24 is a partially enlarged sectional view illustrating a leading portion and a portion in the vicinity thereof in a guide wire according to a fifth embodiment, which is cut at the same position as the line III-III of FIG. 2.

FIG. 25 is a partially enlarged perspective view of a wire rod used in a coil according to the fifth embodiment.

FIG. 26 is a partially enlarged sectional view illustrating a leading portion and a portion in the vicinity thereof in a guide wire according to a sixth embodiment, which is cut at the same position as the line III-III of FIG. 2.

### DESCRIPTION OF EMBODIMENTS

### First Embodiment

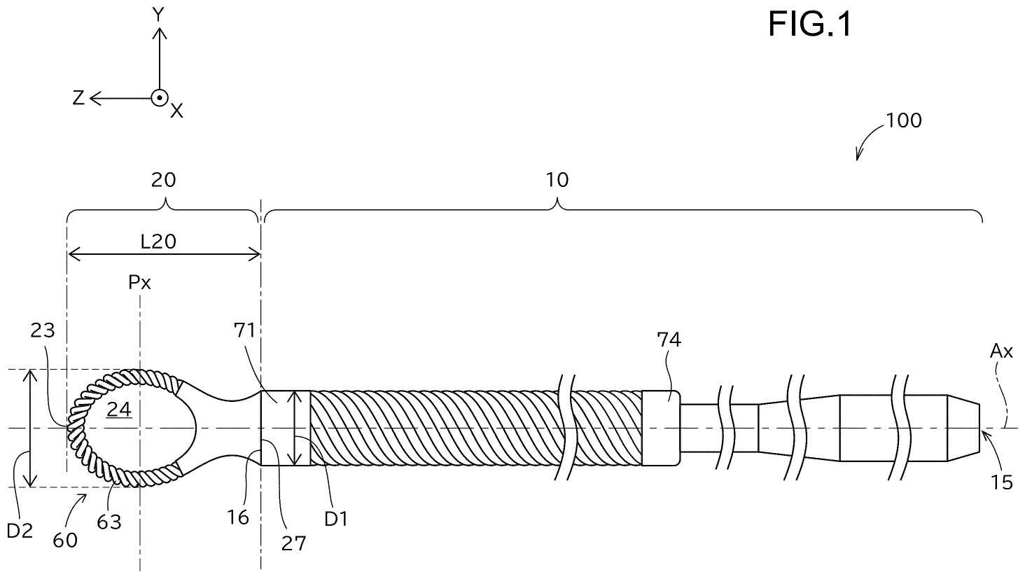

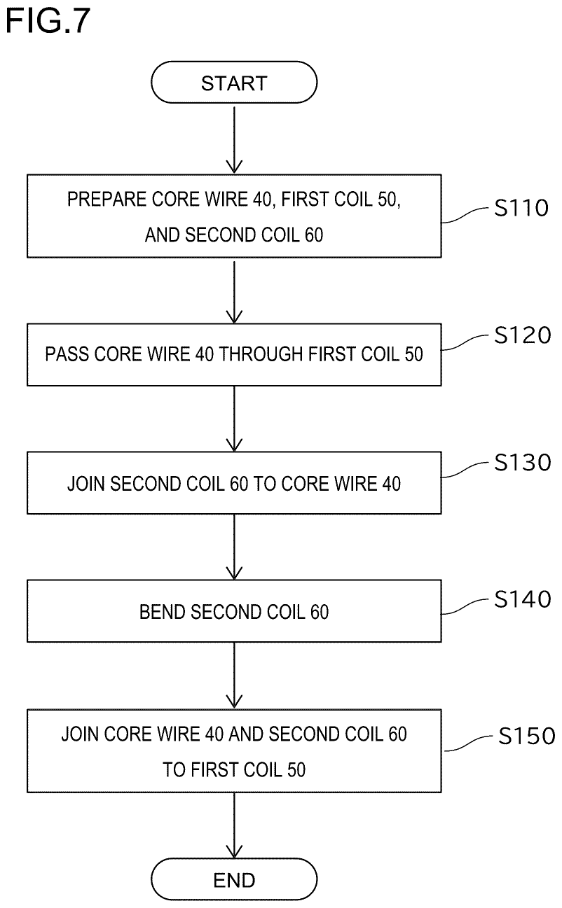

A first embodiment will be described with reference to FIGS. 1 to 12. A guide wire 100 according to the present embodiment is a medical device that is inserted into a living body lumen to treat a lesion in the living body lumen. The living body lumen includes a tubular organ of a human body such as a blood vessel, a digestive tract, a ureter, an organ, or a bile duct. In the guide wire 100, the Z-axis positive direction side is the distal end side to be inserted into a body, and the Z-axis negative direction side is a proximal end side to be operated by a professional such as a doctor. The distal end side is also referred to as a far side, and the proximal end side is also referred to as a near side. In each drawing, a part of the guide wire 100 may be omitted. FIGS. 1, 2 and 3 illustrate the guide wire 100 in a straight state parallel to the Z-axis. The guide wire 100 is flexible enough to be curved. The same applies to the subsequent drawings.

In this specification, with regard to the guide wire 100 and each constituent member thereof, an end on the distal end side is referred to as a “distal end”, the distal end and the vicinity thereof are referred to as a “distal end portion”, an end on the proximal end side is referred to as a “proximal end”, and the proximal end and the vicinity thereof are referred to as a “proximal end portion”. The transverse cross-section of the guide wire 100 and each constituent member thereof means a cross-section perpendicular to the longitudinal direction. The longitudinal cross-section of the guide wire 100 and each constituent component thereof means a cross-section parallel to the center axis in the longitudinal direction. In the guide wire 100 and each constituent component thereof, a direction perpendicular to the longitudinal direction is referred to as a radial direction. The outer diameter of the guide wire 100 and each constituent component thereof means a width in the radial direction.

The guide wire 100 is a medical device to be inserted into a living body lumen such as a blood vessel. The entire length of the guide wire 100 is, for example, 1000 mm or more and 3000 mm or less.

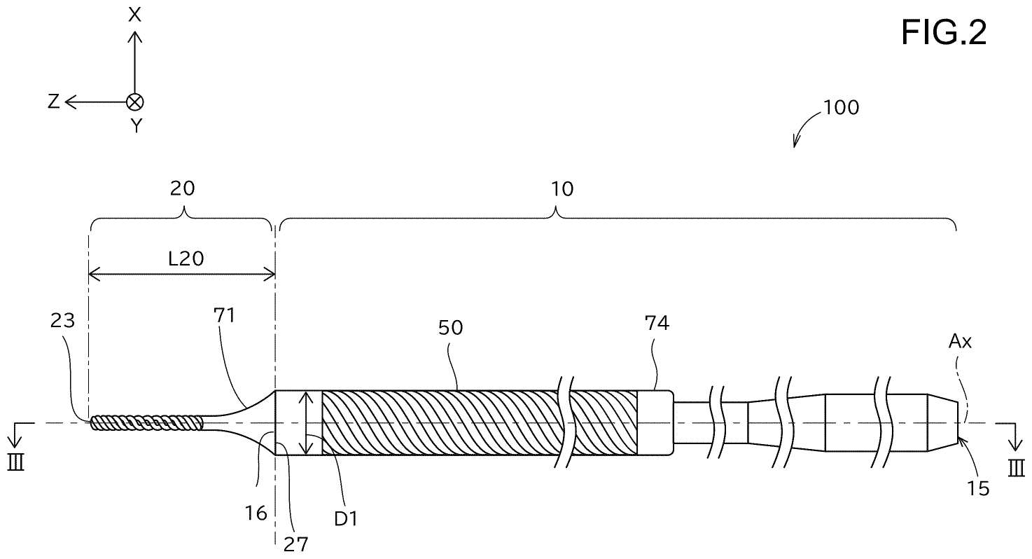

As illustrated in FIGS. 1 and 2, the guide wire 100 includes a main body part 10 and a leading portion 20.

The main body part 10 is an elongated portion extending along a center axis Ax. According to the present embodiment, the center axis Ax of the main body part 10 coincides with the center axis of the guide wire 100. A proximal end 15 of the main body part 10 coincides with the proximal end of the guide wire 100.

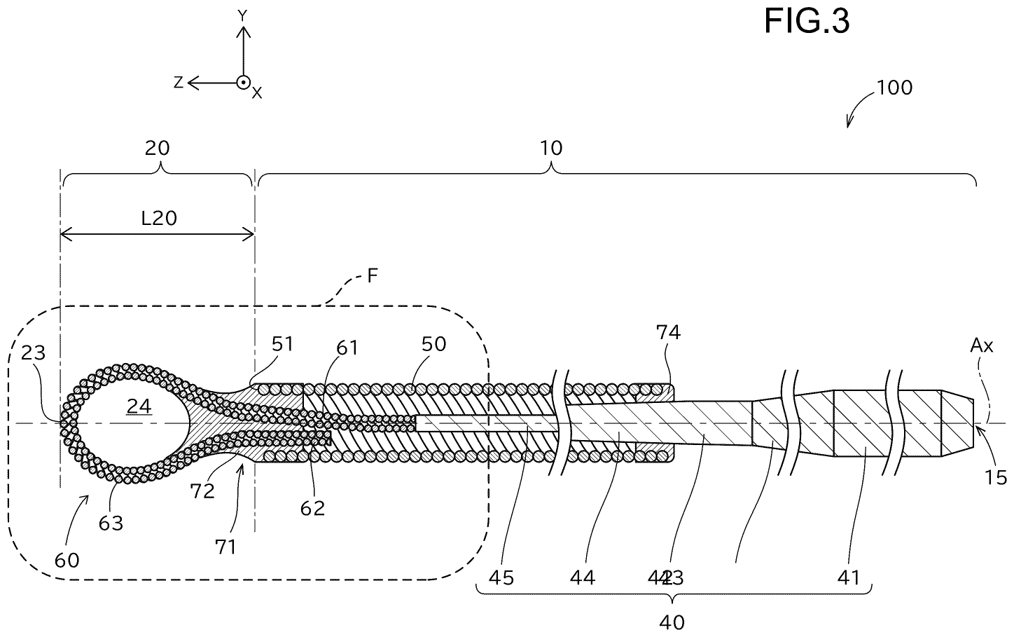

The leading portion 20 may be referred to as a lead portion, a drill portion, a crushing portion, a peeling portion, an entry portion, a peeler, a shaver, or the like. The leading portion 20 has a proximal end 27 connected to a distal end 16 of the main body part 10 and a distal end 23 opposite to the proximal end 27. The distal end 23 of the leading portion 20 coincides with the distal end of the guide wire 100. The surface of the leading portion 20 may have an edge or may have no edge. An edge is a boundary between two surfaces. The leading portion 20 enters a lesion while rotating around the center axis Ax. Entering the lesion by the leading portion 20 may be expressed as crossing or passing through the lesion, drilling the lesion, crushing the lesion, peeling the lesion, raking the lesion, going under the lesion, getting into the lesion, or the like. A length L20 of the leading portion 20 along the center axis Ax is, for example, 0.2 mm or more and 2.0 mm or less. The length L20 of the leading portion 20 may be 0.3 mm or more and 1.5 mm or less, or may be 0.4 mm or more and 1.0 mm or less. As illustrated in FIG. 3, the guide wire 100 includes a core wire 40, a first coil 50, and a second coil 60. The first coil 50 is an example of a tubular body. The second coil 60 is an example of a coil.

The first coil 50 is a hollow cylindrical member in which one or more wire rods are spirally wound. According to the present embodiment, the first coil 50 is a multi-thread coil formed by winding a plurality of wire rods. The outer diameter of the first coil 50 is, for example, 0.1 mm or more and 0.6 mm or less. The outer diameter of the first coil 50 may be 0.2 mm or more and 0.5 mm or less or may be 0.3 mm or more and 0.4 mm or less. The outer diameter of the first coil 50 may be 1.00 mm or more and 2.00 mm or less, may be 1.10 mm or more and 1.65 mm or less, or may be 1.20 mm or more and 1.35 mm or less. According to the present embodiment, the outer diameter of the first coil 50 is constant over the entire length of the first coil 50. The first coil 50 may have a tapered shape in which the outer diameter gradually decreases from the proximal end toward the distal end, or may have a tapered shape in which the outer diameter gradually decreases from the distal end toward the proximal end.

The wire rod forming the first coil 50 may be a single strand or a twisted strand formed by twisting a plurality of strands. According to the present embodiment, the wire rod forming the first coil 50 is a twisted strand.

The material of the first coil 50 is, for example, metal. The material of the first coil 50 may be a material that transmits radiation or may be a material that does not transmit radiation. The material that transmits radiation may be, for example, stainless steels such as SUS302, SUS304, and SUS316, Ni—Ti alloys, and piano wires. The material that does not transmit radiation may be, for example, platinum, gold, tungsten, or an alloy of any of these. The entire first coil 50 may be formed of the same material, or each portion may be formed of a different material.

The core wire 40 is a linear member. The core wire 40 includes a large diameter portion 41, a first tapered portion 42, an intermediate diameter portion 43, a second tapered portion 44, and a small diameter portion 45. The large diameter portion 41, the first tapered portion 42, the intermediate diameter portion 43, the second tapered portion 44, and the small diameter portion 45 are continuous in this order from the proximal end of the core wire 40.

The large diameter portion 41 is a rod-shaped portion having a substantially constant outer diameter. The outer diameter of the large diameter portion 41 is, for example, about 0.2 mm to 3.0 mm. The intermediate diameter portion 43 is a rod-shaped portion that is located closer to the distal end side than the large diameter portion 41 and has a substantially constant outer diameter smaller than the outer diameter of the large diameter portion 41. The first tapered portion 42 is a portion that is located between the large diameter portion 41 and the intermediate diameter portion 43 and has a diameter gradually decreasing from the boundary with the large diameter portion 41 toward the boundary with the intermediate diameter portion 43. The small diameter portion 45 is a rod-shaped portion that is located closer to the distal end side than the intermediate diameter portion 43 and has a substantially constant outer diameter smaller than the outer diameter of the intermediate diameter portion 43. The second tapered portion 44 is a portion that is located between the intermediate diameter portion 43 and the small diameter portion 45 and has a diameter gradually decreasing from the boundary with the intermediate diameter portion 43 toward the boundary with the small diameter portion 45.

The shape of the transverse cross-section of the core wire 40 at each position may be any shape. The shape of the transverse cross-section of the core wire 40 at each position may be a circle, a partial circle, an ellipse, a rectangle, a parallelogram, a trapezoid, a rhombus, or the like. A partial circle is one of the two halves of a circle divided by a chord. The outer edge of the partial circle includes an arc line and a line segment connecting two end portions of the arc line. The partial circle may have, for example, a semicircular shape, a partially circular shape, or an arcuate shape. A semicircular shape is one of two halves of a circle divided by a chord passing through the center of the circle. A partially circular shape is the larger of two halves of a circle divided by a chord that does not pass through the center of the circle. An arcuate shape is the smaller of the two halves divided by a chord that does not pass through the center of the circle. The transverse cross-section of the core wire 40 is not strictly limited to the above-described shapes, and may be substantially the above-described shapes. The shape of the transverse cross-section may be different at each position along the longitudinal direction of the core wire 40.

The material of the core wire 40 is, for example, metal. To be more specific, the material of the core wire 40 may be, for example, stainless steels such as SUS302, SUS304, and SUS316, Ni—Ti alloys, and piano wires. The entire core wire 40 may be formed of the same material, or each portion of the core wire 40 may be formed of a different material.

The core wire 40 is inserted into the inside of the first coil 50. That is, a part of the core wire 40 is covered with the first coil 50. According to the present embodiment, the second tapered portion 44 and the small diameter portion 45 of the core wire 40 are covered with the first coil 50.

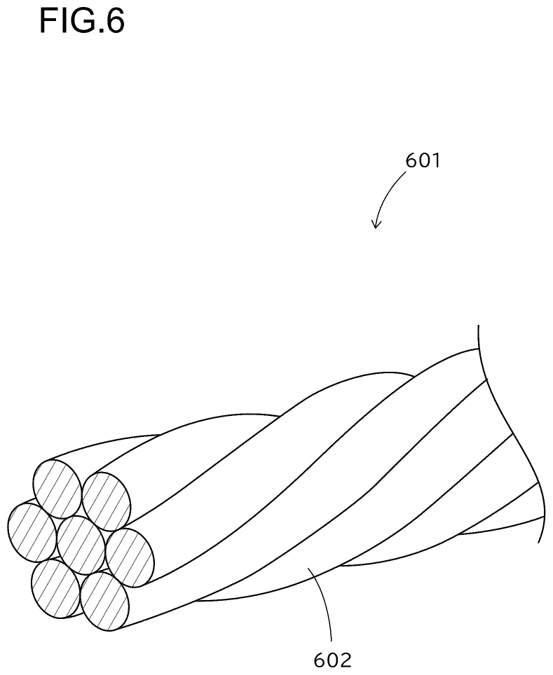

The second coil 60 is a coil in which one or more wire rods 601 are spirally wound. The wire rod forming the second coil 60 may be a single strand or a twisted strand formed by twisting a plurality of strands. According to the present embodiment, as illustrated in FIG. 6, the wire rod 601 forming the second coil 60 is a twisted strand formed by twisting a plurality of strands 602. In the drawings other than FIG. 6, the wire rod 601 is illustrated in a simplified manner in consideration of visibility. According to the present embodiment, the coil diameter of the second coil 60 is substantially constant over the entire length of the second coil 60. The second coil 60 is a densely wound coil that is wound such that adjacent portions of the wire rod 601 are in contact with each other. One end of the second coil 60 is connected to the distal end of the small diameter portion 45.

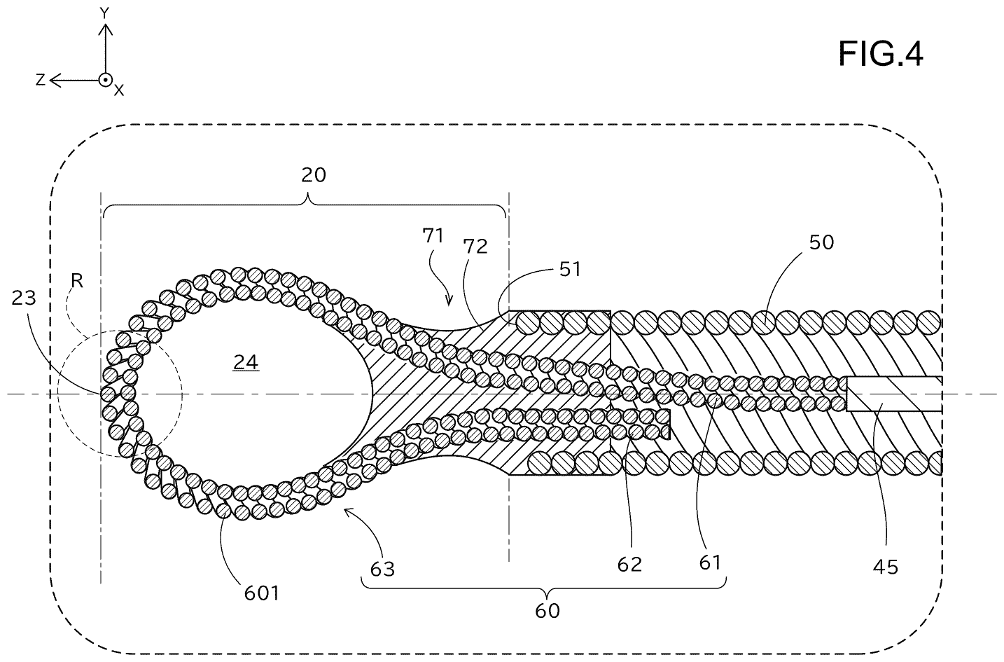

As illustrated in FIGS. 3 and 4, the second coil 60 includes a first end portion 61, a second end portion 62, and a loop portion 63.

The first end portion 61 includes one end of the second coil 60 connected to the small diameter portion 45 and is inserted into the inside of the first coil 50. The second end portion 62 is one of the two end portions of the second coil 60 opposite to the first end portion 61 and is inserted into the inside of the first coil 50. The first end portion 61 and the second end portion 62 extend substantially linearly along the center axis Ax.

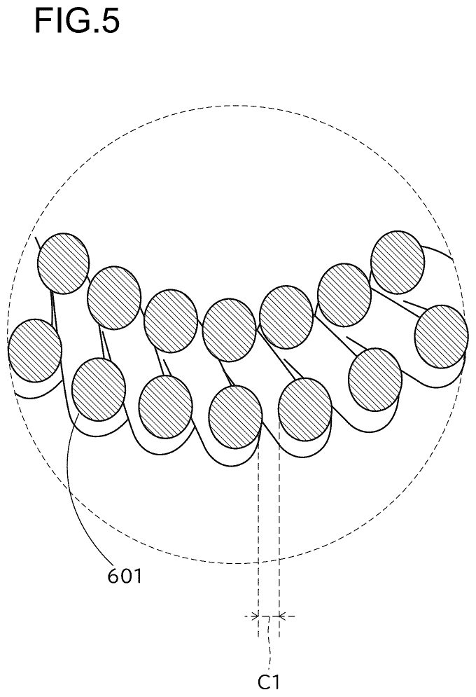

The loop portion 63 is a portion of the second coil 60 between the first end portion 61 and the second end portion 62 and is located closer to the distal end side than the first coil 50. The loop portion 63 is bent in a loop shape. More specifically, the loop portion 63 extends from the distal end of the first end portion 61 toward the distal end while being curved, is folded back at the distal end, and extends toward the proximal end while being curved. In this specification, the term “loop shape” includes not only the case where the loop portion has only a closed ring, but also the case where the loop portion has a shape in which a part of the ring is cut out, and the case where a part of the loop portion extends to the outside of the ring. According to the present embodiment, most of the loop portion 63 except for both end portions forms a shape in which a part of an ellipse having a major axis in a direction along the center axis Ax is cut and both end portions extend substantially linearly toward the proximal end. According to the present embodiment, there is a clearance between both ends of the loop portion 63. Both end portions of the loop portion 63 may be in contact with each other or may intersect each other. The shape of the ring formed by the loop portion may be a circle, a partial circle, an ellipse, a rectangle, a parallelogram, a trapezoid, a rhombus, or the like, or may be a partially distorted shape of these shapes. The space inside the loop portion 63 is a through-hole 24 extending in the X-axis direction. The distal end of the loop portion 63 coincides with the distal end 23 of the leading portion 20. Since the loop portion 63 has a curved shape, as illustrated in FIG. 5, there is a clearance C1 between adjacent portions of the wire rod 601 on the outer peripheral side of the loop portion 63. According to the present embodiment, the curvature of the loop portion 63 is larger in the vicinity of the distal end 23 of the leading portion 20 than in other portions, and the clearance C1 is also relatively large in the vicinity of the distal end 23.

The material of the second coil 60 is, for example, metal. The material of the second coil 60 may be a material that transmits radiation or a material that does not transmit radiation. The material that transmits radiation may be, for example, stainless steels such as SUS302, SUS304, and SUS316, nickel, Ni—Ti alloys, and piano wire. The material that does not transmit radiation may be, for example, platinum, gold, tungsten, or an alloy of any of these. According to the present embodiment, the material of the second coil 60 is a material that does not transmit radiation. The entire second coil 60 may be formed of the same material, or each portion of the second coil 60 may be formed of a different material.

The distal end portion of the first coil 50 is joined to the second coil 60 with a distal end side joining member 71. The distal end side joining member 71 is an example of a joining member. A part of the distal end side joining member 71 enters the inside of the first coil 50 and joins a part of the first end portion 61, a part of the second end portion 62, and the distal end portion of the first coil 50. Another part of the distal end side joining member 71 serves as a reinforcing portion 72 protruding to the distal end side from the distal end 51 of the first coil 50. The reinforcing portion 72 covers both end portions of the loop portion 63. The reinforcing portion 72 reinforces a connection portion of the leading portion 20 with the main body part 10. A connection portion between the second coil 60 and the core wire 40 is inside the first coil 50. The connection portion between the second coil 60 and the core wire 40 may be embedded inside the distal end side joining member 71 or may be located closer to the proximal end side than the distal end side joining member 71. The proximal end portion of the first coil 50 is joined to the core wire 40 with a proximal end side joining member 74. The first coil 50 may be joined to the core wire 40 via a joining member formed at another position. The material of the distal end side joining member 71 and the proximal end side joining member 74 is, for example, solder, a brazing material, or an adhesive. The solder may be, for example, an Au—Sn alloy, an Sn—Ag alloy, an Sn—Pb alloy, or a Pb—Ag alloy. The brazing material may be, for example, an aluminum alloy brazing material, a silver brazing material, or a gold brazing material. The adhesive may be, for example, an epoxy-based adhesive.

The main body part 10 includes the core wire 40, the first coil 50, the first end portion 61, the second end portion 62, a portion of the distal end side joining member 71 excluding the reinforcing portion 72, and the proximal end side joining member 74. The leading portion 20 includes the loop portion 63 and the reinforcing portion 72.

As illustrated in FIG. 1, the outer diameter of the leading portion 20 changes along the center axis Ax as viewed in the X-axis direction. Specifically, the outer diameter of the leading portion 20 at the proximal end 27 of the leading portion 20 is substantially the same as a maximum outer diameter D1 of the distal end 16 of the main body part 10. The outer diameter of the leading portion 20 gradually decreases from the proximal end 27 of the leading portion 20 toward the distal end side, then gradually increases to reach a maximum outer diameter D2 at a maximum outer diameter position Px, and gradually decreases from the maximum outer diameter position Px to the distal end 23 of the leading portion 20. The maximum outer diameter D2 of the leading portion 20 is, for example, 0.2 mm or more and 1.0 mm or less. The maximum outer diameter D2 of the leading portion 20 may be 0.3 mm or more and 0.8 mm or less, or may be 0.4 mm or more and 0.6 mm or less. The maximum outer diameter D2 of the leading portion 20 may be 1.00 mm or more and 3.00 mm or less, may be 1.20 mm or more and 2.50 mm or less, or may be 1.50 mm or more and 2.00 mm or less.

The maximum outer diameter D2 of the leading portion 20 is measured as follows. The measurer observes the guide wire 100 from the side. The side is in the Y-axis direction according to the present embodiment. The measurer searches for the angle at which the near side portion and the far side portion of the second coil 60 overlap each other and the far side portion is invisible. For example, when both of the following two conditions are satisfied, the measurer searches for the angle at which the portion between the second end portion 62 and the distal end 23 is invisible. The first condition is that the portion between the first end portion 61 and the distal end 23 is on the near side. The second condition is that the portion between the second end portion 62 and the distal end 23 is on the far side. The non-visibility of the portion between the second end portion 62 and the distal end 23 is caused by the overlap between the portion between the first end portion 61 and the distal end 23 and the portion between the second end portion 62 and the distal end 23. Subsequently, the measurer captures the guide wire 100 using a microscope along the viewpoint rotated by 90 degrees around the center axis Ax from the viewpoint at this time. The measurer sets the capture magnification of the microscope to 200 times or more. The measurer measures the outer diameter of the leading portion 20 at three measurement positions where the leading portion 20 is considered to have the maximum outer diameter D2 on the captured image. To be specific, at each measurement position, the measurer draws a pair of straight lines that pass through a pair of end portions of the leading portion 20 in the outer diameter direction, are perpendicular to the outer diameter direction, and are parallel to each other and measures the interval between the pair of straight lines. The measurer adopts the maximum value of the measurement results at the three measurement positions as the maximum outer diameter D2 of the leading portion 20.

As illustrated in FIG. 1, the leading portion 20 has the maximum outer diameter D2 that is larger than the maximum outer diameter D1 of the distal end 16 of the main body part 10. According to the present embodiment, the maximum outer diameter D2 of the leading portion 20 coincides with the maximum outer diameter of the loop portion 63.

Next, an example of a method for manufacturing the guide wire 100 will be described.

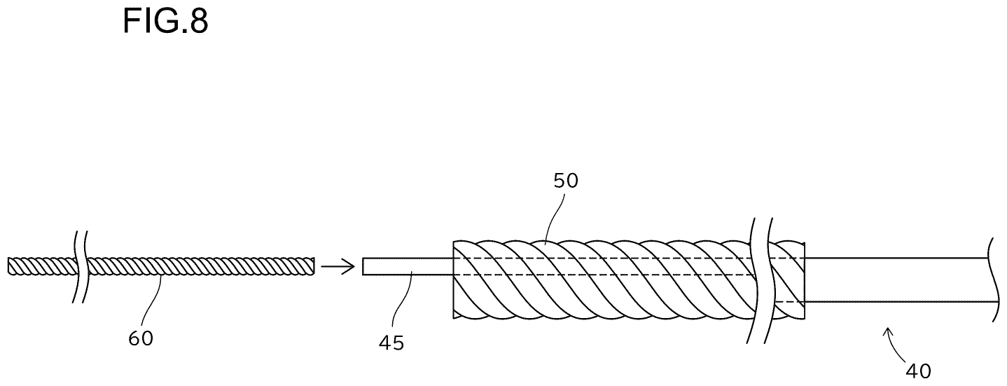

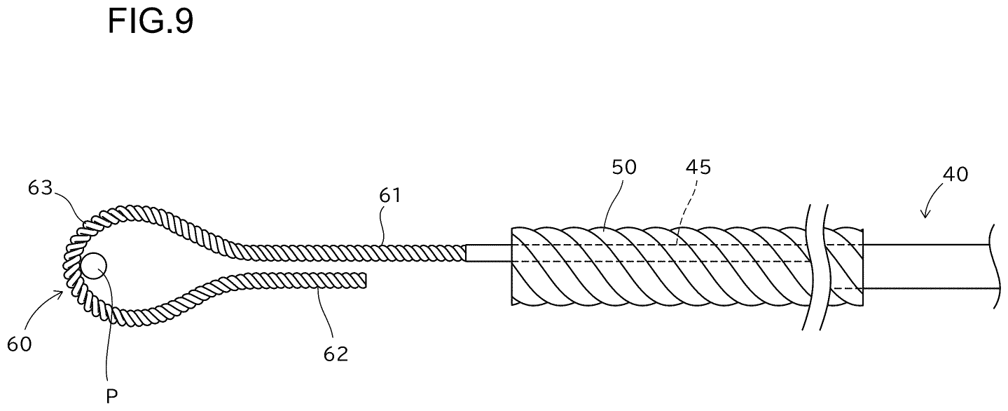

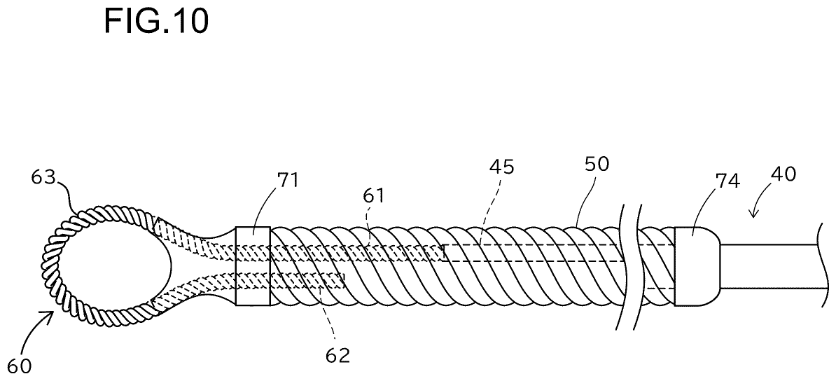

First, the core wire 40, the first coil 50, and the second coil 60 are prepared as separate members (S110, FIG. 8). Subsequently, the core wire 40 is passed through the first coil 50 and is located such that the distal end portion protrudes from the first coil 50 to the distal end side (S120, FIG. 8). Subsequently, the proximal end of the second coil 60 is joined to the distal end of the core wire 40 by, for example, welding (S130, FIG. 8). At the time of joining, the second coil 60 is not bent and is in a straight state. After the joining, the second coil 60 is bent into a loop shape using a pin P (S140, FIG. 9). After the bending process, the first end portion 61 and the second end portion 62 of the second coil 60 are inserted into the inside of the first coil 50 and are joined to the distal end portion of the first coil 50 by the distal end side joining member 71. Subsequently, the core wire 40 is joined to the proximal end portion of the first coil 50 by the proximal end side joining member 74 (S150, FIG. 10). For example, the guide wire 100 according to the present embodiment is manufactured by the above-described process.

In the method for manufacturing the guide wire 100 according to the present embodiment, the core wire 40 and the second coil 60, which is formed as a member separate from the core wire 40, are joined. With this configuration, the guide wire 100 in which the leading portion 20 includes the loop portion 63 formed by the second coil 60 is easily manufactured.

In the method for manufacturing the guide wire 100, as the length of the second coil 60 and the length of the core wire 40 are changed, the properties of the guide wire 100 are changed. That is, by appropriately setting the length of the second coil 60 and the length of the core wire 40, the guide wire 100 having desired properties can be obtained.

Next, an example of a treatment method using a catheter 120 including the guide wire 100 will be described.

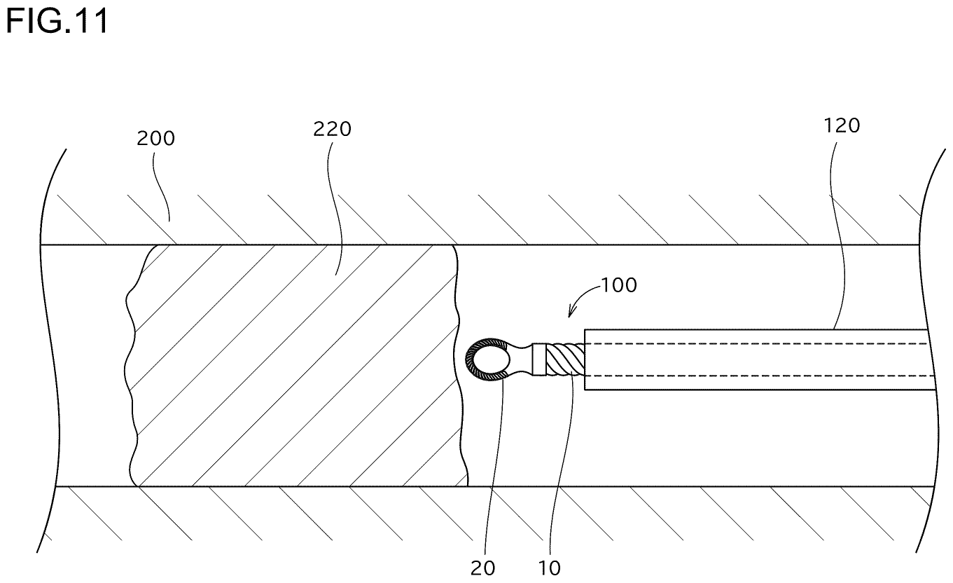

The professional inserts a preceding guide wire (not illustrated) into a blood vessel 200 and advances the preceding guide wire to the front of a lesion 220. The preceding guide wire is referred to as a workhorse guide wire, a first choice guide wire, or the like. Subsequently, the professional inserts the catheter 120 into the blood vessel 200 along the preceding guide wire. The professional advances the catheter 120 to the front of the lesion 220 in the blood vessel 200. Subsequently, the professional removes the preceding guide wire from the blood vessel 200. Then, the professional inserts the guide wire 100 into the catheter 120, which is inserted into the blood vessel 200, with the leading portion 20 at the head (FIG. 11). The professional advances the guide wire 100 to the front of the lesion 220 in the blood vessel 200. When the guide wire 100 is advanced, the guide wire 100 may be rotated around the center axis Ax or may not be rotated.

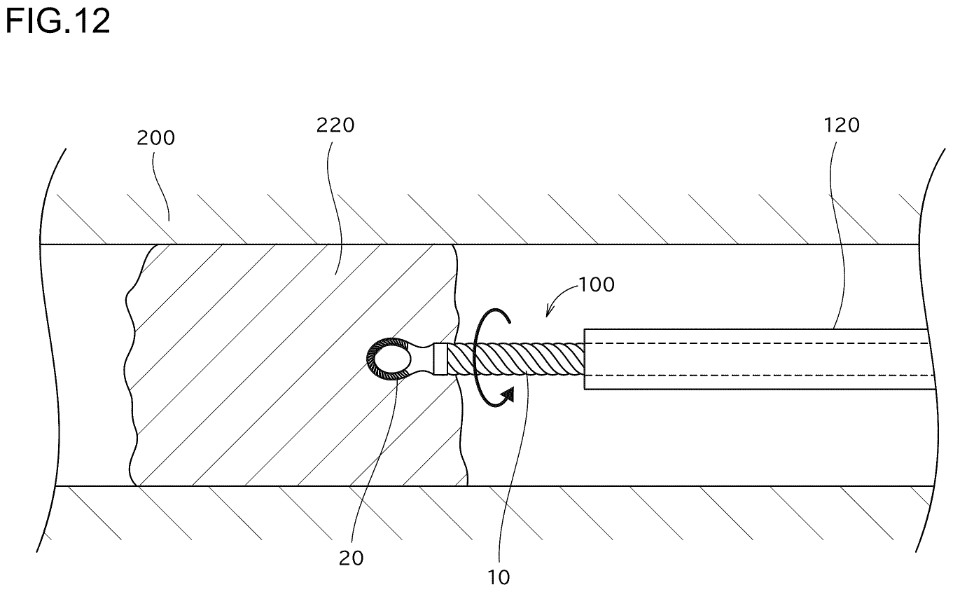

Subsequently, the professional advances the leading portion 20 into the lesion 220 by advancing the guide wire 100 to the distal end side while rotating the guide wire 100 (FIG. 12). When the professional grips the proximal end portion of the guide wire 100 and rotates the guide wire 100 around the center axis Ax, the leading portion 20 located at the distal end portion of the guide wire 100 also rotates around the center axis Ax. The leading portion 20 rotating in the lesion 220 excavates so as to cut the lesion 220.

The leading portion 20 has a certain degree of flexibility so as not to damage the blood vessel 200 when the guide wire 100 is advanced inside the blood vessel 200. According to the present embodiment, the loop portion 63 is formed by the second coil 60 in which the wire rod 601 is wound. The loop portion 63 has high flexibility and is easily deformed by a small force. That is, since the loop portion 63 is easily deformed when the loop portion 63 abuts against the inner wall of the blood vessel 200, the load applied from the leading portion 20 to the inner wall of the blood vessel 200 is reduced. This results in an improvement of safety. In addition, the wire rod 601 is a twisted strand formed by twisting the plurality of strands 602. When the wire rod 601 is a twisted strand, flexibility is higher and disconnection is less likely to occur than in the case where the wire rod is a single strand. Therefore, safety is further enhanced.

When the guide wire 100 passes through the lesion 220, the leading portion 20 is required to advance while efficiently excavating the lesion 220. According to the present embodiment, since the second coil 60 is a densely wound coil, a constant rigidity is secured in the loop portion 63. Therefore, the lesion 220 is efficiently cut. Further, there is the clearance C1 between adjacent portions of the wire rod 601 forming the loop portion 63. When the lesion 220 enters the clearance C1, the wire rod 601 is easily caught by the lesion 220 so that the lesion 220 is efficiently cut. As a result, the passing performance of the leading portion 20 with respect to the lesion 220 is improved. In addition, the maximum outer diameter D2 of the leading portion 20 is larger than the maximum outer diameter D1 of the distal end 16 of the main body part 10. That is, there is a relatively large clearance between the distal end 16 of the main body part 10 and the inner wall of the blood vessel 200. Therefore, small pieces of the lesion 220 generated by the contact between the leading portion 20 and the lesion 220 are smoothly discharged from the distal end side to the proximal end side of the main body part 10 through the clearance. As a result, the small pieces of the lesion 220 are prevented from obstructing the excavation by the leading portion 20, and the passing performance of the leading portion 20 with respect to the lesion 220 is improved.

In order for the leading portion 20 to efficiently excavate the lesion 220, the guide wire 100 has high torquability. The torquability is a performance of smoothly transmitting torque to the distal end portion when the proximal end portion is rotationally operated by a professional such as a doctor. According to the present embodiment, the main body part 10 includes the core wire 40 connected to the second coil 60. With this configuration, when the proximal end portion of the guide wire 100 is rotationally operated by the professional, the torque is smoothly transmitted to the second coil 60 via the core wire 40. As a result, desirable torquability is ensured.

When the leading portion 20 is advanced into the lesion 220 as described above, for example, imaging of a blood vessel 200 using a radioscopic apparatus is used. The radioscopic apparatus is, for example, a fluoroscope. According to the present embodiment, the second coil 60 is formed of a material that does not transmit radiation and has radiopacity. Therefore, the position of the leading portion 20 is easily visually recognized under radioscopy.

As described above, the guide wire 100 according to the present embodiment includes the main body part 10 and the leading portion 20 that is connected to the distal end of the main body part 10 and enters the lesion 220. The leading portion 20 includes the loop portion 63 that is formed by the second coil 60 in which the wire rod 601 is wound and that has a loop shape. With this configuration, safety is improved.

According to the present embodiment, the main body part 10 includes the core wire 40 connected to the second coil 60. With this configuration, desirable torquability is ensured.

According to the present embodiment, the main body part 10 further includes the first coil 50. A connection portion between the second coil 60 and the core wire 40 is located inside the first coil 50. With this configuration, the connection portion between the second coil 60 and the core wire 40 is protected by the first coil 50.

According to the present embodiment, the maximum outer diameter D2 of the leading portion 20 is larger than the maximum outer diameter D1 of the distal end 16 of the main body part 10. With this configuration, the passing performance of the leading portion 20 with respect to the lesion 220 is improved.

According to the present embodiment, the second coil 60 has radiopacity. With this configuration, visibility of the leading portion 20 under radioscopy is improved.

According to the present embodiment, at the distal end 23 of the leading portion 20, there is the clearance C1 between adjacent portions of the wire rod 601. With this configuration, the passing performance of the leading portion 20 with respect to the lesion 220 is improved.

According to the present embodiment, the wire rod 601 is a twisted strand formed by twisting the plurality of strands 602. With this configuration, safety is further enhanced.

According to the present embodiment, the second coil 60 is a densely wound coil that is wound such that adjacent portions of the wire rod 601 are in contact with each other. With this configuration, the passing performance of the leading portion 20 with respect to the lesion 220 is improved.

In the method for manufacturing the guide wire 100 according to the present embodiment, the core wire 40 and the second coil 60 formed as a member separate from the core wire 40 are joined. With this configuration, the guide wire 100 including the loop portion 63 formed by the second coil 60 is easily manufactured. In addition, it is easy to impart desired properties to the guide wire 100.

### Second Embodiment

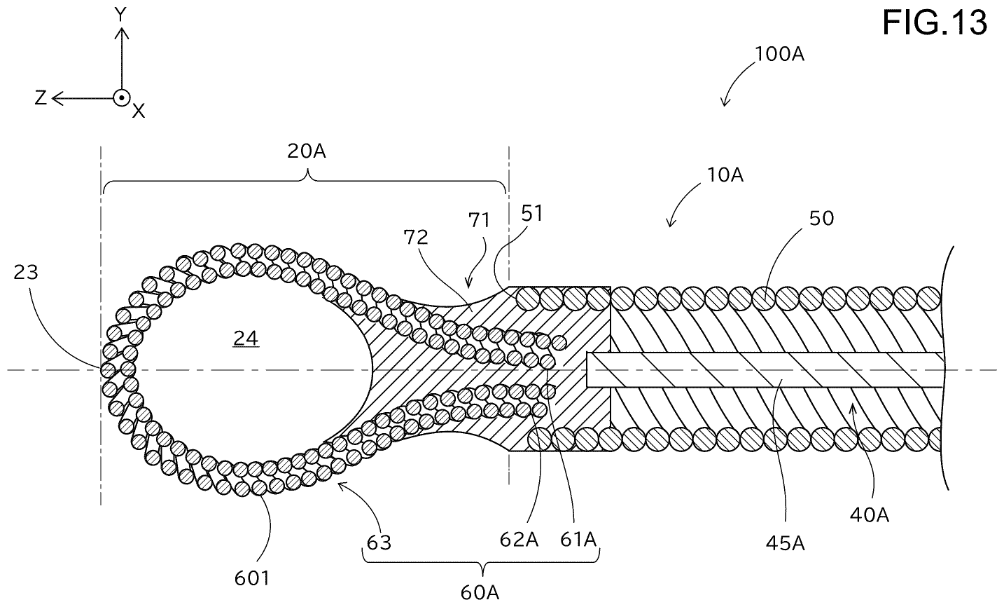

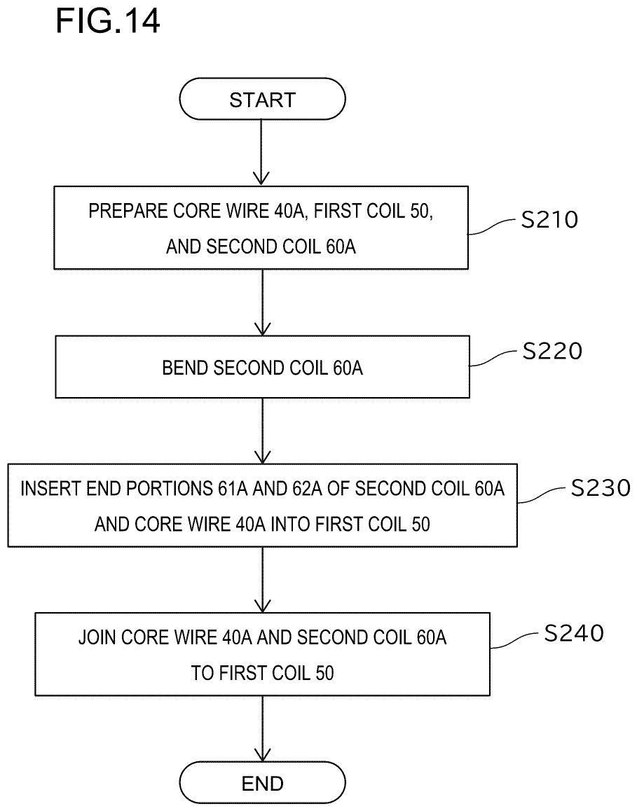

A second embodiment will be described with reference to FIGS. 13 to 16. In a guide wire 100A according to the present embodiment, some of the configurations of a core wire 40A and a second coil 60A are different from those of the first embodiment. According to the present embodiment, the same components as those of the first embodiment are denoted by the same reference numerals, and description thereof will be omitted.

As illustrated in FIG. 13, the guide wire 100A according to the present embodiment includes the core wire 40A, the first coil 50, and the second coil 60A. The second coil 60A is an example of a coil.

The core wire 40A has the same configuration as in the first embodiment, except that a distal end of a small diameter portion 45A is closer to the distal end 51 of the first coil 50 than in the first embodiment. The second coil 60A has the same configuration as in the first embodiment except that a proximal end of a first end portion 61A and a proximal end of a second end portion 62A are closer to the distal end 51 of the first coil 50 than in the first embodiment.

The distal end portion of the first coil 50 is joined to the core wire 40A by the distal end side joining member 71. A part of the distal end side joining member 71 enters the inside of the first coil 50 and joins the first end portion 61A, the second end portion 62A, and the distal end portion of the first coil 50. The first end portion 61A, the second end portion 62A, and the distal end portion of the small diameter portion 45A are embedded inside the distal end side joining member 71. Another part of the distal end side joining member 71 serves as the reinforcing portion 72 protruding to the distal end side from the distal end 51 of the first coil 50. The reinforcing portion 72 covers both end portions of the loop portion 63.

A main body part 10A includes the core wire 40A, the first coil 50, the first end portion 61A, the second end portion 62A, a portion of the distal end side joining member 71 excluding the reinforcing portion 72, and the proximal end side joining member 74. A leading portion 20A includes the loop portion 63 and the reinforcing portion 72.

Next, an example of a method for manufacturing the guide wire 100A will be described.

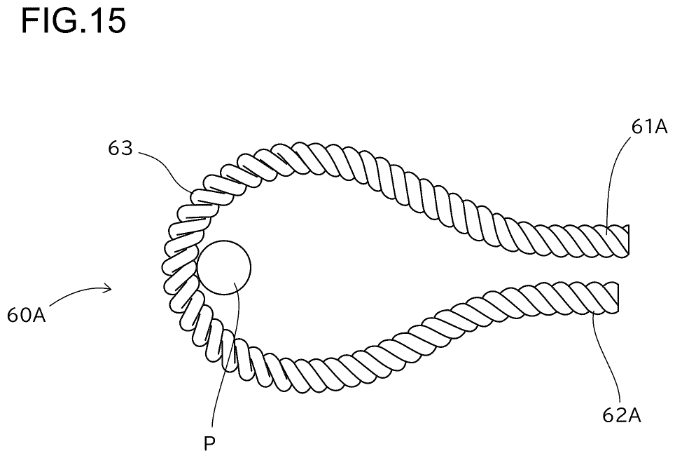

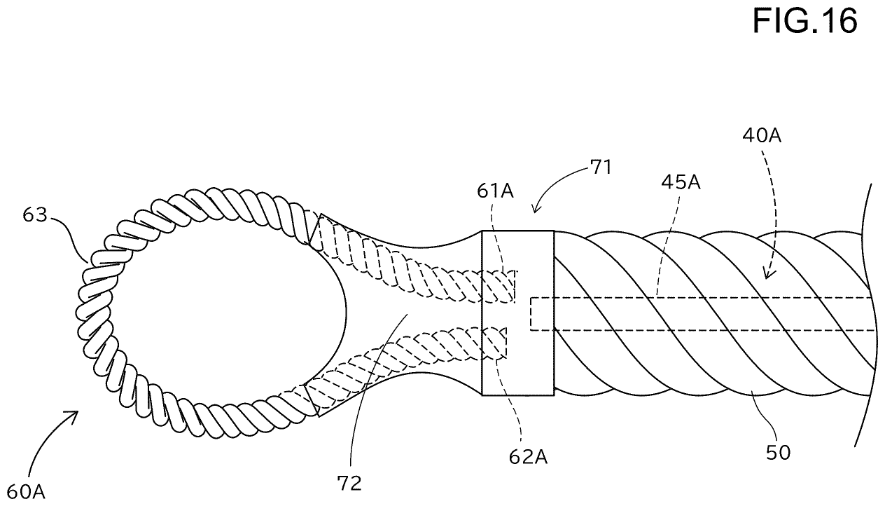

First, the core wire 40A, the first coil 50, and the second coil 60A are prepared as separate members (S210). Subsequently, the second coil 60A is bent into a loop shape using the pin P (S220, FIG. 15). After the bending process, the first end portion 61A and the second end portion 62A of the second coil 60A and the core wire 40A are inserted into the inside of the first coil 50 (S230, FIG. 16). Subsequently, the first end portion 61A, the second end portion 62A, and the distal end portion of the core wire 40A are joined to the distal end portion of the first coil 50 by the distal end side joining member 71. Subsequently, the core wire 40A is joined to the proximal end portion of the first coil 50 by the proximal end side joining member 74 (S240, FIG. 16). For example, the guide wire 100A according to the present embodiment is manufactured by the above-described process.

As described above, the guide wire 100A according to the present embodiment includes the main body part 10A and the leading portion 20A, similarly to the first embodiment. The leading portion 20A includes the loop portion 63 that is formed by the second coil 60A in which the wire rod 601 is wound and that has a loop shape. With this configuration, safety is improved.

The guide wire 100A according to the present embodiment further includes the distal end side joining member 71 that joins the first coil 50 and the core wire 40A. The first end portion 61A and the second end portion 62A of the second coil 60A are embedded inside the distal end side joining member 71. With this configuration, only the portion on the distal end side of the guide wire 100A is formed by the second coil 60A. Therefore, the flexibility of the design of the main body part 10A is increased. In addition, the core wire 40A can be located up to the vicinity of the distal end of the main body part 10A. Therefore, desirable torquability is secured.

In the method for manufacturing the guide wire 100 according to the present embodiment, the core wire 40A, the second coil 60A formed as a member separate from the core wire 40A, and the first coil 50 are joined by the distal end side joining member 71. With this configuration, the guide wire 100A including the loop portion 63 formed by the second coil 60A is easily manufactured.

### Third Embodiment

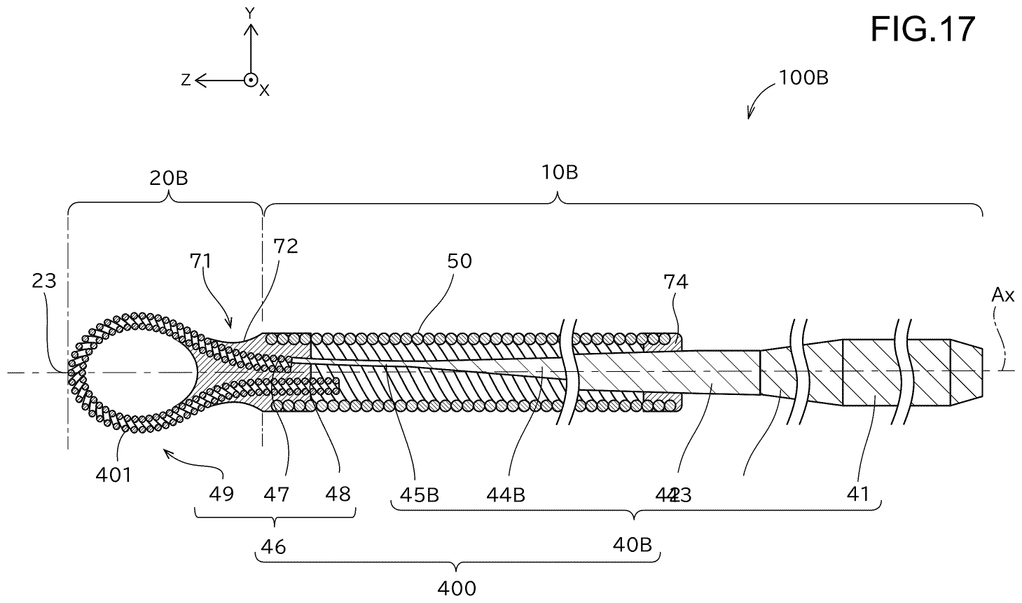

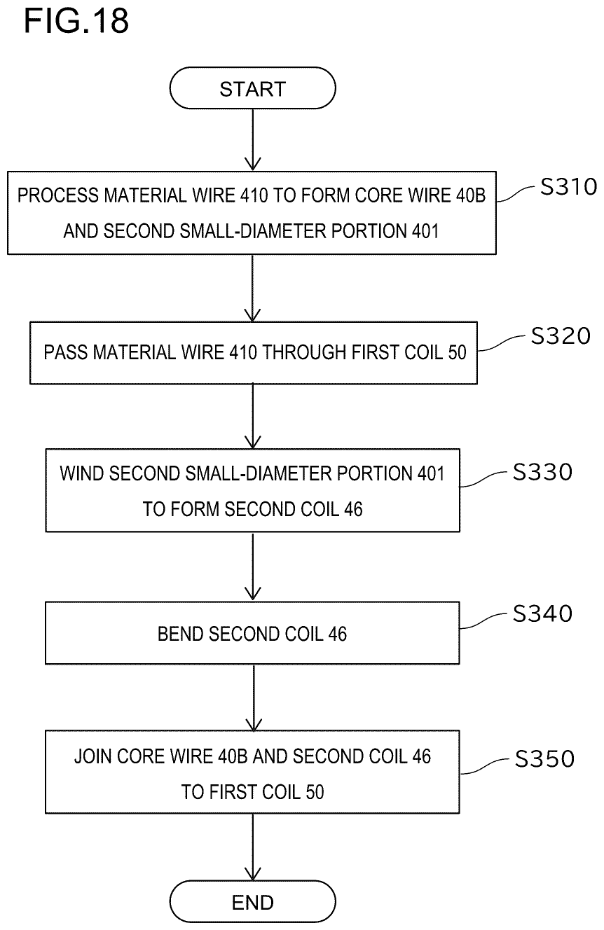

A third embodiment will be described with reference to FIGS. 17 to 22. A guide wire 100B according to the present embodiment is different from that of the first embodiment in that a second coil 46 including a loop portion 49 and a core wire 40B are integrally formed as a single member. The second coil 46 is an example of a coil. According to the present embodiment, the same components as those of the first embodiment are denoted by the same reference numerals, and description thereof will be omitted.

As illustrated in FIG. 17, the guide wire 100B according to the present embodiment includes a wire member 400 and the first coil 50. The wire member 400 includes the core wire 40B and the second coil 46.

The core wire 40B includes the large diameter portion 41, the first tapered portion 42, the intermediate diameter portion 43, a second tapered portion 44B, a first small diameter portion 45B, and the second coil 46. The large diameter portion 41, the first tapered portion 42, the intermediate diameter portion 43, the second tapered portion 44B, and the first small diameter portion 45B are continuous in this order from the proximal end of the core wire 40B.

The portion from the large diameter portion 41 to the intermediate diameter portion 43 is the same as in the first embodiment. The first small diameter portion 45B is a thin linear portion that is located closer to the distal end side than the intermediate diameter portion 43 and has a substantially constant outer diameter smaller than the outer diameter of the intermediate diameter portion 43. The second tapered portion 44B is a portion that is located between the intermediate diameter portion 43 and the first small diameter portion 45B and has a diameter gradually decreasing from the boundary with the intermediate diameter portion 43 toward the boundary with the first small diameter portion 45B.

The second coil 46 is formed by spirally winding a second small diameter portion 401 of the wire member 400, which has a thin linear shape and is continuous with the distal end of the first small diameter portion 45B. The second small diameter portion 401 is an example of a wire rod and a small diameter portion. According to the present embodiment, the wire diameter of the second small diameter portion 401 is equal to the outer diameter of the first small diameter portion 45B. The second coil 46 may be a densely wound coil that is wound such that adjacent portions of the second small diameter portion 401 are in contact with each other, or may be a loosely wound coil that is wound such that there is a clearance between adjacent portions of the second small diameter portion 401. According to the present embodiment, the coil diameter of the second coil 46 is substantially constant over the entire length of the second coil 46.

The second coil 46 includes a first end portion 47, a second end portion 48, and the loop portion 49. The first end portion 47 is an end portion of the second coil 46 that is continuous with the first small diameter portion 45B and is inserted into the inside of the first coil 50. The second end portion 48 is one of the two end portions of the second coil 46 opposite to the first end portion 47 and is inserted into the inside of the first coil 50.

The loop portion 49 is a portion of the second coil 46 between the first end portion 47 and the second end portion 48 and is located closer to the distal end side than the first coil 50. As in the first embodiment, the loop portion 49 is bent into a loop shape. The detailed shape of the loop portion 49 is the same as that of the loop portion 63 according to the first embodiment.

The distal end portion of the first coil 50 is joined to the wire member 400 by the distal end side joining member 71. A part of the distal end side joining member 71 enters the inside of the first coil 50 and joins the first end portion 47, the second end portion 48, and the distal end portion of the first coil 50. Another part of the distal end side joining member 71 serves as the reinforcing portion 72 protruding to the distal end side from the first coil 50. The reinforcing portion 72 covers both end portions of the loop portion 49. A connection portion between the second coil 46 and the core wire 40B is inside the first coil 50. The connection portion between the second coil 46 and the core wire 40B may be embedded inside the distal end side joining member 71 or may be located closer to the proximal end side of the distal end side joining member 71.

The main body part 10B includes the core wire 40B, the first coil 50, the first end portion 47, the second end portion 48, a portion of the distal end side joining member 71 excluding the reinforcing portion 72, and the proximal end side joining member 74. A leading portion 20B includes the loop portion 49 and the reinforcing portion 72.

Next, an example of a method for manufacturing the guide wire 100B will be described.

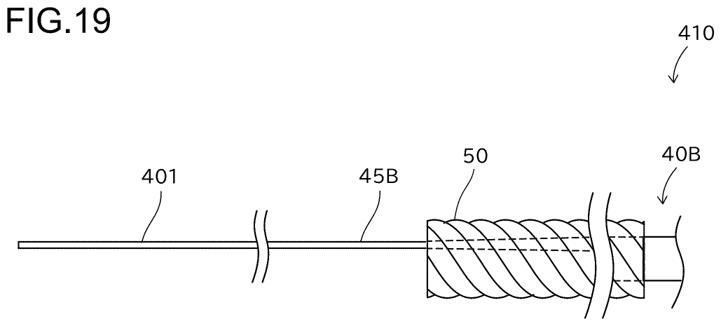

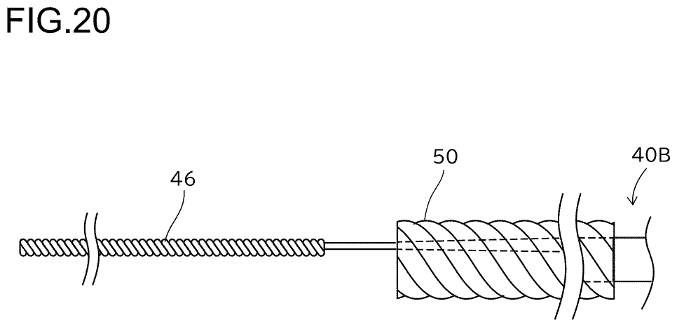

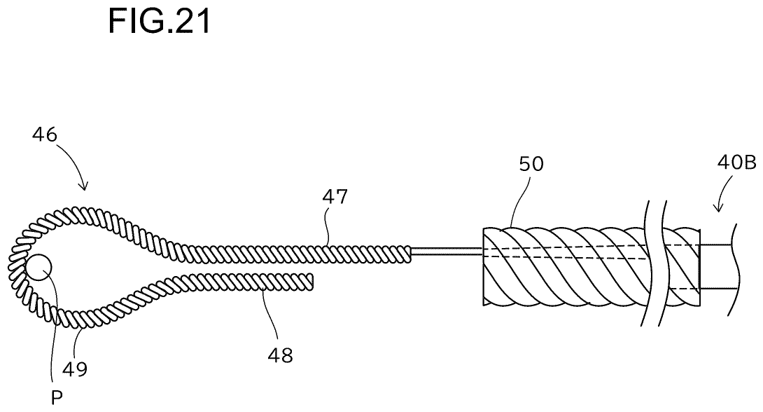

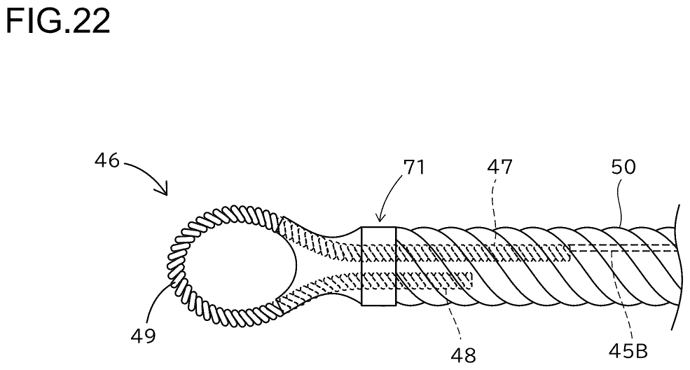

First, a material wire 410 serving as a material of the core wire 40B is prepared. The material wire 410 is subjected to, for example, a drawing process to form the core wire 40B and the second small diameter portion 401 continuous with the core wire 40B (S310, FIG. 19). Subsequently, the material wire 410 after the drawing process is passed through the first coil 50 and is located such that the second small diameter portion 401 protrudes from the first coil 50 to the distal end side (S320, FIG. 19). Subsequently, the second small diameter portion 401 is wound to form the second coil 46 (S330, FIG. 20). Subsequently, the second coil 46 is bent into a loop shape using the pin P (S340, FIG. 21). After the bending process, the first end portion 47 and the second end portion 48 of the second coil 46 are inserted into the inside of the first coil 50 and are joined to the distal end portion of the first coil 50 by the distal end side joining member 71. Subsequently, the core wire 40B is joined to the proximal end portion of the first coil 50 by the proximal end side joining member 74 (S350, FIG. 22). For example, the guide wire 100 according to the present embodiment is manufactured by the above-described process.

As described above, the guide wire 100B according to the present embodiment includes the main body part 10B and the leading portion 20B, similarly to the first embodiment. The leading portion 20B includes the loop portion 49 that is formed by the second coil 46 in which the second small diameter portion 401 is wound and that has a loop shape. With this configuration, safety is improved.

In the method for manufacturing the guide wire 100B according to the present embodiment, the material wire 410 serving as the material of the core wire 40B is processed to form the core wire 40B and the second small diameter portion 401 continuous with the core wire 40B. Subsequently, the second coil 46 is formed by winding the second small diameter portion 401. Subsequently, the leading portion 20B is formed by bending the second coil 46 into a loop shape. With this configuration, since it is not necessary to join the core wire 40B and the second coil 46, the manufacturing process is simplified.

### Fourth Embodiment

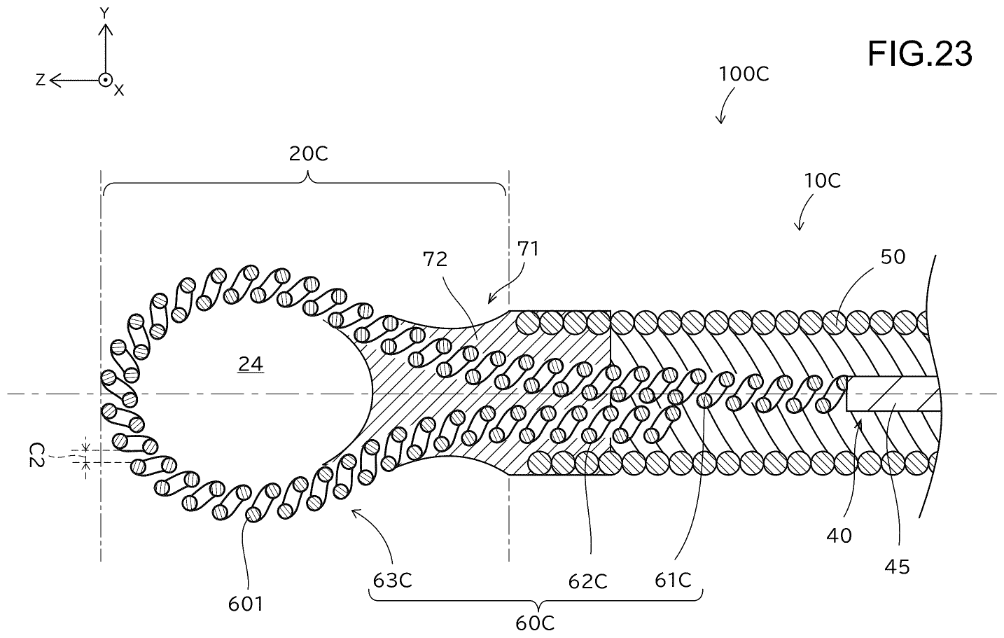

A fourth embodiment will be described with reference to FIG. 23. In a guide wire 100C according to the present embodiment, the configuration of a second coil 60C is different from that of the first embodiment. The second coil 60C is an example of a coil. According to the present embodiment, the same components as those of the first embodiment are denoted by the same reference numerals, and description thereof will be omitted.

The second coil 60C according to the present embodiment is the same as that of the first embodiment except that the second coil 60C is a loosely wound coil that is wound to have a clearance between adjacent portions of a wire rod 601. As in the first embodiment, the second coil 60C includes a first end portion 61C, a second end portion 62C, and a loop portion 63C. Since the second coil 60C is a loosely wound coil, the size of a clearance C2 between adjacent portions of the wire rod 601 is larger than that in the first embodiment in which the second coil 60 is a densely wound coil.

Similarly to the first embodiment, the guide wire 100C according to the present embodiment includes a main body part 10C and a leading portion 20C. The leading portion 20C includes the loop portion 63C that is formed by the second coil 60C in which the wire rod 601 is wound and that has a loop shape. With this configuration, safety is improved.

According to the present embodiment, the second coil 60C is a loosely wound coil that is wound to have a clearance between adjacent portions of the wire rod 601.

Therefore, the flexibility of the loop portion 63C is further enhanced. In addition, the clearance C2 between adjacent portions of the wire rod 601 is relatively large, and the wire rod 601 is likely to be caught by the lesion 220. As a result, both the safety and the passing performance through the lesion 220 are achieved.

### Fifth Embodiment

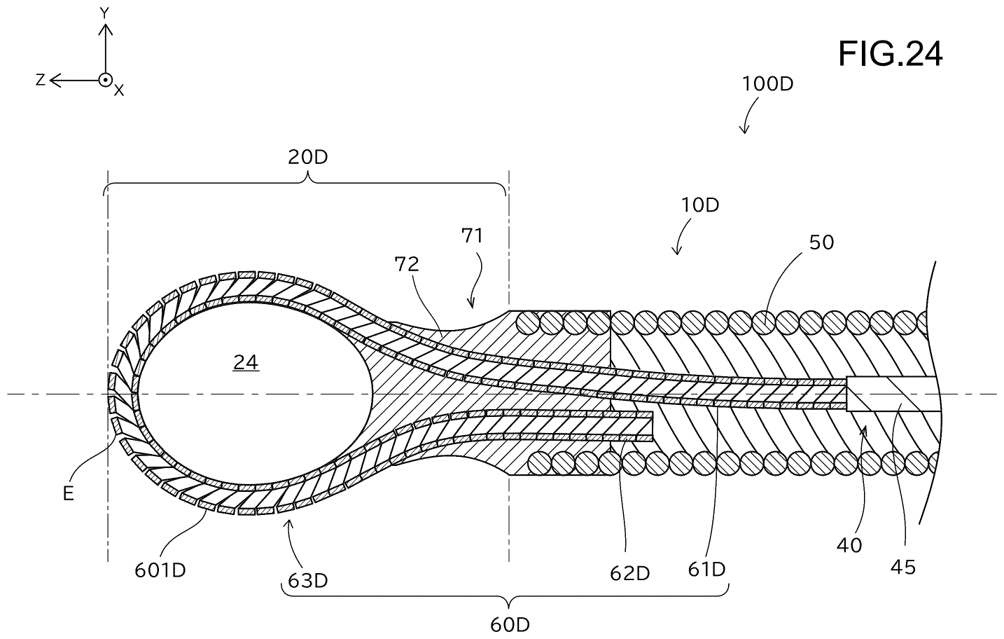

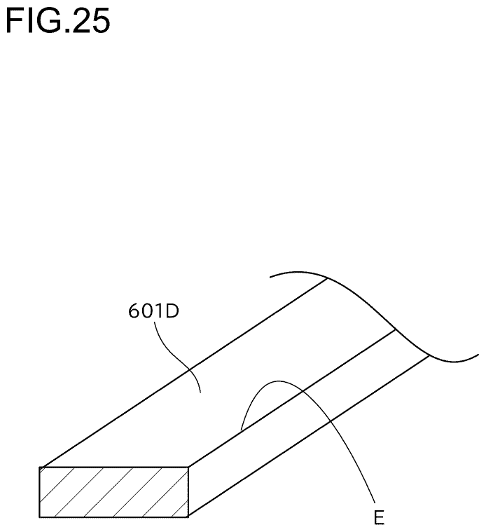

A fifth embodiment will be described with reference to FIGS. 24 and 25. In a guide wire 100D according to the present embodiment, the configuration of a second coil 60D is different from that of the first embodiment. The second coil 60D is an example of a coil. According to the present embodiment, the same components as those of the first embodiment are denoted by the same reference numerals, and description thereof will be omitted.

The second coil 60D according to the present embodiment is the same as that of the first embodiment except that a wire rod 601D is a single strand and the cross-sectional shape of the wire rod 601D is rectangular. As in the first embodiment, the second coil 60D includes a first end portion 61D, a second end portion 62D, and a loop portion 63D.

Similarly to the first embodiment, the guide wire 100D according to the present embodiment includes a main body part 10D and a leading portion 20D. The leading portion 20D includes a loop portion 63D that is formed by the second coil 60D in which the wire rod 601D is wound and that has a loop shape. With this configuration, safety is improved.

According to the present embodiment, since the cross-sectional shape of the wire rod 601D in the second coil 60D is rectangular, an edge E, which is a boundary between two surfaces of the wire rod 601D, exists on the surface of the loop portion 63D.

Therefore, the wire rod 601D is likely to be caught by the lesion 220. As a result, the passing performance of the leading portion 20D with respect to the lesion 220 is improved. In addition, the second coil 60D, in which the cross-sectional shape of the wire rod 601D is rectangular, has the same rigidity and is smaller in size than a coil in which the cross-sectional shape of the wire rod is circular. Therefore, the leading portion 20D is made compact.

### Sixth Embodiment

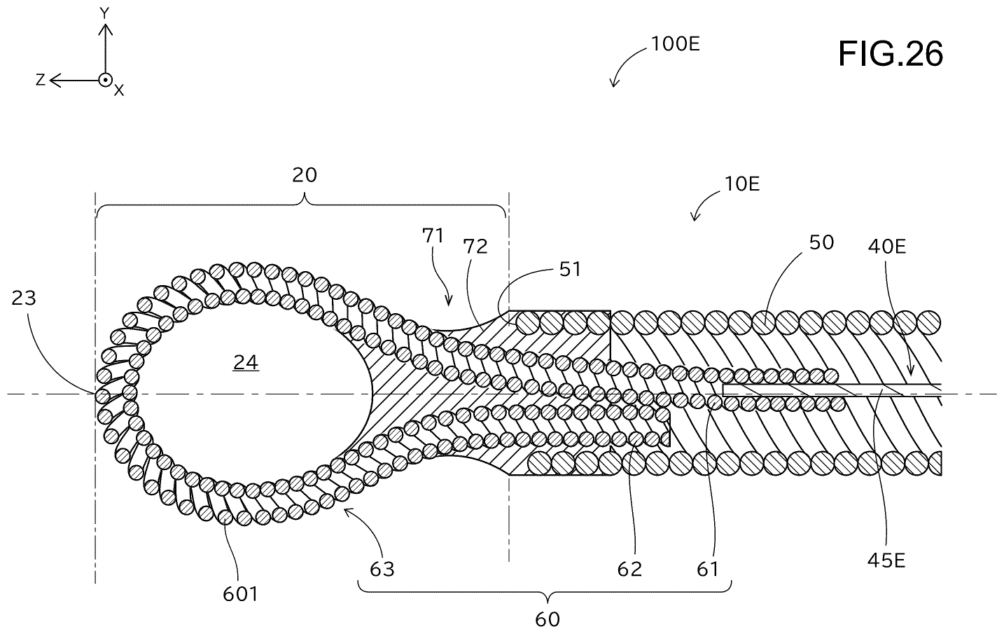

A sixth embodiment will be described with reference to FIG. 26. In a guide wire 100E according to the present embodiment, the configuration of a connection portion between a second coil 60 and a core wire 40E is different from that of the first embodiment. According to the present embodiment, the same components as those of the first embodiment are denoted by the same reference numerals, and description thereof will be omitted.

As illustrated in FIG. 26, the guide wire 100E according to the present embodiment includes the core wire 40E, the first coil 50, and the second coil 60.

The core wire 40E according to the present embodiment has the same configuration as that of the first embodiment except that the outer diameter of a small diameter portion 45E is smaller than the inside diameter of the second coil 60. A distal end portion of the small diameter portion 45E coincides with the distal end portion of the core wire 40E. The distal end portion of the small diameter portion 45E is located inside the first end portion 61. The distal end portion of the small diameter portion 45E is joined to the first end portion 61. A method for joining the small diameter portion 45E and the first end portion 61 may be, for example, welding or soldering.

As in the first embodiment, the guide wire 100E according to the present embodiment includes a main body part 10E and the leading portion 20. The leading portion 20 includes the loop portion 63 that is formed by the second coil 60 in which the wire rod 601 is wound and that has a loop shape. With this configuration, safety is improved.

According to the present embodiment, the distal end portion of the core wire 40E is located inside the second coil 60, and the second coil 60 and the distal end portion of the core wire 40E are joined. With this configuration, the joining strength between the second coil 60 and the core wire 40E is increased.

### Modification

The technology disclosed herein is not limited to the above-described embodiments, and can be modified into various forms without departing from the gist thereof, and for example, the following modifications are also possible.
(1) The guide wire may not include the first coil.
(2) The method for connecting the main body part and the coil may be different from the joining using the distal end side joining member 71. For example, the coil may be fixed to the main body part by covering the main body part and the coil with a tubular member and crimping the tubular member.
(3) In the first embodiment and the third embodiment, it has been described that the connection portion between the second coil 60 and the core wire 40 (40B) may be embedded inside the distal end side joining member 71 or may be located closer to the proximal end side than the distal end side joining member 71. This also applies to the fourth embodiment, the fifth embodiment, and the sixth embodiment.
(4) In the above embodiment, the guide wire 100 for treating a lesion in a blood vessel has been described as an example. The technology disclosed herein is similarly applicable to medical devices in general for treating lesions in a living body lumen.

1. A medical device comprising:
a main body part; and
a leading portion including a loop-shaped loop portion that is formed by a coil, wherein the coil is formed by a spirally wound wire rod;
wherein the leading portion is connected to a distal end of the main body part and is configured to enter a lesion.

2. The medical device according to claim 1, wherein the main body part includes a core wire connected to the coil.

3. The medical device according to claim 2, wherein
the main body part further includes a tubular body, and
a connection portion between the coil and the core wire is located inside the tubular body.

4. The medical device according to claim 3, further comprising a joining member that joins the tubular body and the core wire, wherein
an end portion of the coil is embedded inside the joining member.

5. The medical device according to claim 2, wherein a distal end portion of the core wire is located inside the coil, and the coil and the distal end portion of the core wire are joined.

6. The medical device according to claim 2, wherein the coil and the core wire are integrally formed as a single member.

7. The medical device according to claim 1, wherein a maximum outer diameter of the leading portion is larger than a maximum outer diameter of the distal end of the main body part.

8. The medical device according to claim 1, wherein the coil has radiopacity.

9. The medical device according to claim 1, wherein, in a distal end of the leading portion, there is a clearance between adjacent portions of the spirally wound wire rod.

10. The medical device according to claim 1, wherein the spirally wound wire rod is a twisted strand formed by twisting a plurality of strands.

11. The medical device according to claim 1, wherein a transverse cross-section of the spirally wound wire rod is rectangular.

12. The medical device according claim 1, wherein the coil is a loosely wound coil in which the spirally wound wire rod is wound to have a clearance between adjacent portions of the spirally wound wire rod.

13. The medical device according to claim 1, wherein the coil is a densely wound coil in which the spirally wound wire rod is wound such that adjacent portions of the spirally wound wire rod are in contact with each other.

14. A method for manufacturing a medical device including
a main body part including a core wire connected to a coil, and
a leading portion including a loop portion that is formed by at least a part of the coil, wherein the coil is formed by a spirally wound wire rod;
wherein the leading portion is connected to a distal end of the main body part and is configured to enter a lesion,
the method comprising joining the core wire and the coil formed as a member separate from the core wire.

15. The method for manufacturing the medical device according to claim 14, the main body part further including a tubular body,
the method further comprising joining the core wire, the coil, and the tubular body by a joining member.
